(19) United States
(12) Patent Application Publication
(10) Pub. No.: US 2026/0215840 A1
(43) Pub. Date: Jul. 30, 2026

(54) CROSS-PROBE ABLATION SPLIT HUB AND METHODS OF USING THE SAME

(71) Applicant: Medtronic Europe Sàrl, Tolochenaz (CH)

(72) Inventor: Calin Druma, San Jose, CA (US)

(21) Appl. No.: 19/145,752

(22) PCT Filed: Dec. 19, 2023
(86) PCT No.: PCT/EP2023/086607

§ 371 Date: Jul. 3, 2025

Related U.S. Application Data
(60) Provisional application No. 63/437,063, filed on Jan. 4, 2023.

(51) Int. Cl. A61B 18/14; A61B 18/00; A61B 18/12; G01K 7/02

(52) U.S. Cl. CPC A61B 18/1477; A61B 18/1206; G01K 7/02; A61B 2018/00023; A61B 2018/00172; A61B 2018/00577; A61B 2018/00779; A61B 2018/00791; A61B 2018/00875

(57) ABSTRACT

A device according to at least one embodiment of the present disclose includes a first port and a second port each configured to respectively connect to a first probe and a second probe, where the first probe and the second probe are configured to perform bipolar ablation; and a third port and a fourth port each configured to respectively connect to a third probe and a fourth probe, where the third probe and the fourth probe are configured to perform bipolar ablation.

### BACKGROUND

The present disclosure is generally directed to surgical tools, and relates more particularly to surgical tools capable of ablating anatomical tissue.

Various surgical tools are often required to successfully complete surgical procedures. Certain types of tools may be required for different procedures. Some surgical procedures utilize applied electrical current for ablation. Bipolar ablation may occur when current passes through anatomical tissue positioned between two electrodes.

### BRIEF SUMMARY

Example aspects of the present disclosure include:

A device according to at least one embodiment of the present disclosure comprises: a first port and a second port each configured to respectively connect to a first probe and a second probe, wherein the first probe and the second probe are configured to perform bipolar ablation; and a third port and a fourth port each configured to respectively connect to a third probe and a fourth probe, wherein the third probe and the fourth probe are configured to perform bipolar ablation.

Any of the features herein, further comprising: an output port that connects two or more of the first port, the second port, the third port, and the fourth port to a radio frequency (RF) generator; and a thermocouple port that is releasably connectable to a thermocouple.

Any of the features herein, wherein the RF generator comprises a display.

Any of the features herein, wherein the display is configured to render information received from at least one of the first probe, the second probe, the third probe, the fourth probe, and the thermocouple port.

Any of the features herein, wherein the information comprises at least one of a temperature measurement, a constant power measurement, a constant impedance measurement, a modulated power measurement, and a modulated impedance measurement.

Any of the features herein, wherein the display is configured to render information associated with at least one of a cross-ablation mode, a coaxial mode, and a retract mode.

Any of the features herein, wherein the first probe, when in the coaxial mode, performs bipolar ablation with a first and a second electrode each coaxially disposed on the first probe.

Any of the features herein, wherein two or more of the first probe, the second probe, the third probe, and the fourth probe are fluidically connected to a coolant pump.

A system according to at least one embodiment of the present disclosure comprises: a radio frequency (RF) generator; and a hub port device comprising: a first port and a second port each configured to respectively connect to a first probe and a second probe, wherein the first probe and the second probe are configured to perform bipolar ablation; and a third port and a fourth port each configured to respectively connect to a third probe and a fourth probe, wherein the third probe and the fourth probe are configured to perform bipolar ablation.

Any of the features herein, wherein the hub port device further comprises: a thermocouple port to which a thermocouple is releasably connectable.

Any of the features herein, wherein an output port connects the first port, the second port, the third port, and the fourth port to the RF generator.

Any of the features herein, further comprising: a display in communication with the RF generator and the hub port device.

Any of the features herein, wherein the display is configured to render information received from at least one of the first probe, the second probe, the third probe, and the fourth probe.

Any of the features herein, wherein the information comprises at least one of a temperature measurement, a constant power measurement, a constant impedance measurement, a modulated power measurement, and a modulated impedance measurement.

Any of the features herein, wherein the display is configured to render at least one of a cross-ablation mode, a coaxial mode, and a retract mode.

Any of the features herein, wherein the first probe, when in the coaxial mode, performs bipolar ablation with a first and a second electrode each coaxially disposed on the first probe.

Any of the features herein, further comprising: a coolant pump fluidically connectable to two or more of the first probe, the second probe, the third probe, and the fourth probe.

A system according to at least one embodiment of the present disclosure comprises: a radio frequency (RF) generator; a display; a hub port device comprising: a first port and a second port each configured to respectively connect to a first probe and a second probe, wherein the first probe and the second probe are configured to perform bipolar ablation; and a third port and a fourth port each configured to respectively connect to a third probe and a fourth probe, wherein the third probe and the fourth probe are configured to perform bipolar ablation; a processor; and a memory storing data thereon that, when processed by the processor, cause the processor to: cause the RF generator to generate an RF current passed into the first probe and the second probe; receive measurement data associated with the first probe and the second probe; and render the measurement data to the display.

Any of the features herein, wherein the measurement data comprises at least one of a temperature measurement, a constant power measurement, a constant impedance measurement, a modulated power measurement, and a modulated impedance measurement.

Any of the features herein, wherein the display is configured to render information about a coaxial mode, and wherein the first probe, when in the coaxial mode, performs RF ablation between a first and a second electrode each coaxially disposed on the first probe.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

Further disclosed herein is a device that includes a first port and a second port each configured to respectively connect to a first probe and a second probe, where the first probe and the second probe are configured to perform bipolar ablation; and a third port and a fourth port each configured to respectively connect to a third probe and a fourth probe, where the third probe and the fourth probe are configured to perform bipolar ablation.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view of aspects of a surgical probe according to at least one embodiment of the present disclosure;

FIG. 1B is a detailed perspective view of a distal end of the surgical probe according to at least one embodiment of the present disclosure;

FIG. 1C is another detailed perspective view of the distal end of the surgical probe according to at least one embodiment of the present disclosure;

FIG. 1D is a cross-sectional plan view of a housing of the surgical probe according to at least one embodiment of the present disclosure;

FIG. 1E is a cross-sectional perspective view of the housing of the surgical probe according to at least one embodiment of the present disclosure;

FIG. 2A is a diagram of a bipolar ablation system according to at least one embodiment of the present disclosure;

FIG. 2B is a diagram of bipolar probes performing coaxial ablation according to at least one embodiment of the present disclosure;

FIG. 2C is a diagram of bipolar probes performing cross-probe ablation according to at least one embodiment of the present disclosure;

FIG. 3 is a block diagram of a system according to at least one embodiment of the present disclosure;

FIG. 4 is a schematic of a cable hub according to at least one embodiment of the present disclosure;

FIG. 5A is an image of a screen with coaxial ablation information according to at least one embodiment of the present disclosure;

FIG. 5B is another image of the screen with coaxial ablation information according to at least one embodiment of the present disclosure;

FIG. 5C is another image of the screen with coaxial ablation information according to at least one embodiment of the present disclosure;

FIG. 5D is an image of a screen with cross-probe ablation information according to at least one embodiment of the present disclosure;

FIG. 5E is another image of the screen with cross-probe ablation information according to at least one embodiment of the present disclosure;

FIG. 5F is an image of a screen with retract ablation information according to at least one embodiment of the present disclosure;

FIG. 5G is another image of the screen with retract ablation information according to at least one embodiment of the present disclosure;

FIG. 6 is a flowchart according to at least one embodiment of the present disclosure; and

FIG. 7 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Ablation systems may use two bipolar probes (e.g., radiofrequency (RF) probes) that are placed transpedicular to a diseased vertebral body in order to ablate the diseased tissue. Depending on the size of the vertebra, the resulting ablation shape presents a depression at the proximal aspect of the lesion. In other words, a portion of the diseased tissue may remain untreated even after the tissue surrounding the bipolar probes has been ablated. Many vertebral tumors form diseased tissue on the posterior wall of the vertebra. It is desirable to ablate this posterior wall tissue with probes using the same settings as those used in ablating the anterior portion of the vertebra.

According to at least one embodiment of the present disclosure, a system includes bipolar probes that deliver a straight posterior line ablation implementing one or more algorithms and monitored by a controller or other device.

According to at least one embodiment of the present disclosure, while in coaxial ablation mode, energy is delivered between the tip (e.g., a distal) electrode and ring (e.g., a proximal) electrode on a single probe. In embodiments with four probes, any combination of the four tips and rings may be activated to perform ablation (e.g., the tip and ring of the first electrode performs ablation, the tip and ring of the second electrode performs ablation, etc.).

According to at least one embodiment of the present disclosure, while in a cross-probe mode, energy (e.g., current, RF energy, etc.) is delivered between the tip electrodes and the ring electrodes of a pair of probes. For example, a first and second probe may perform cross-ablation, where current is passed from the tip electrode of the first probe to the tip electrode of the second probe (or vice versa), or where current is passed from the ring electrode of the first probe to the ring electrode of the second probe (or vice versa). In embodiments with four probes, a single or both probe pairs (e.g., the first and second probes perform cross-ablation and the third and fourth probes perform a separate cross-ablation) may be utilized to ablate anatomical tissue.

Embodiments of the present disclosure provide technical solutions to one or more of the problems of (1) insufficient ablation zones when performing ablations, (2) temperature and power monitoring when performing ablations, and (3) charring of anatomical tissues when performing ablations.

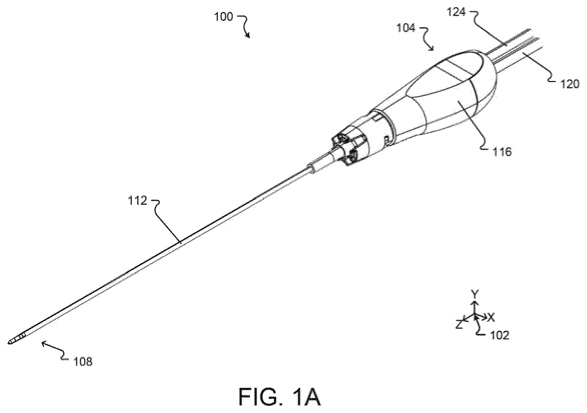

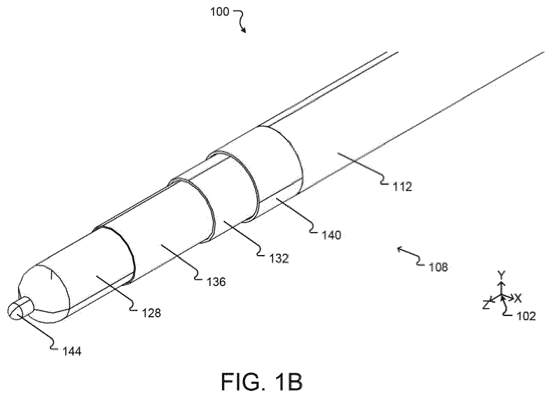

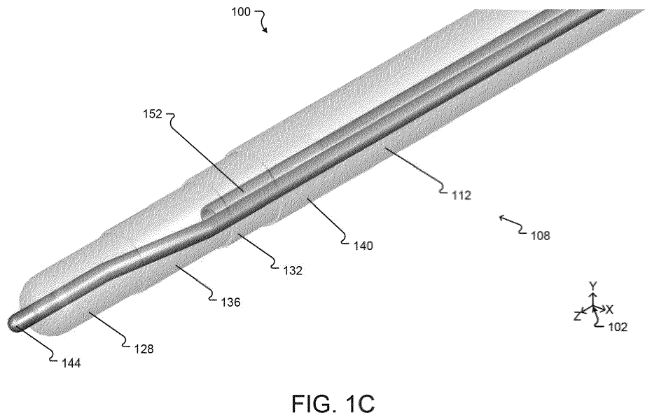

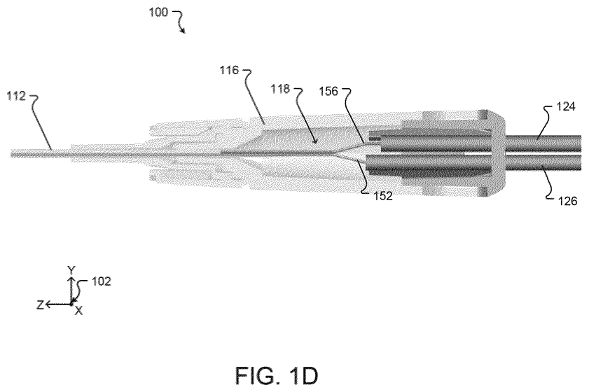

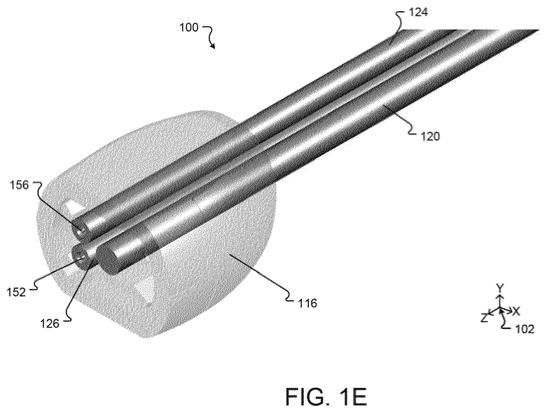

Turning first to FIGS. 1A-1E, aspects of a probe 100 according to at least one embodiment of the present disclosure are shown. The probe 100 may be used to perform coaxial ablation, cross-probe ablation (when paired with another probe), and/or to carry out one or more other aspects of one or more of the methods disclosed herein. The probe 100 extends from a proximal end 104 to a distal end 108 and includes an elongated shaft 112, a clamshell 116, an electric cable 120, a coolant output conduit 124, and a coolant input conduit 126.

In some embodiments, reference may be made to dimensions, angles, directions, relative positions, and/or movements associated with one or more components of the probe 100 with respect to a coordinate system 102. The coordinate system 102, as shown in the accompanying figures, includes three-dimensions comprising an X-axis, a Y-axis, and a Z-axis. Additionally or alternatively, the coordinate system 102 may be used to define planes (e.g., the XY-plane, the XZ-plane, and the YZ-plane) of the probe 100. These planes may be disposed orthogonal, or at 90 degrees, to one another. While the origin of the coordinate system 102 may be placed at any point on or near the probe 100, for the purposes of description, the axes of the coordinate system 102 are disposed along the same directions from figure to figure. Additionally or alternatively, the directionality of the X-axis, Y-axis, and Z-axis may be flipped, as noted with negative directionality (e.g., the negative X-axis direction is the opposite direction of the X-axis direction illustrated by the direction of the associated arrow).

The clamshell 116 may provide a location for the physician to grip the probe 100 when inserting or extracted the probe 100 to or from a surgical site. The clamshell 116 may also provide a housing for the elongated shaft 112, the electric cable 120, the coolant output conduit 124, and/or the coolant input conduit 126, and/or for any of the other components of the probe 100 (or portions thereof). As illustrated in FIGS. 1D and 1E, the clamshell 116 may provide an interior cavity 118 through which the electrical cable 120 may extend. The electric cable 120 may be or comprise an insulative tubing containing the current-carrying wiring. The current-carrying wiring connects to a first electrode 128 and a second electrode 132, forming a circuit through which current can pass. The wiring may carry current generated by a generator or other power source (discussed below) into the probe 100. In some embodiments, the clamshell 116 may encompass portions of electrodes of the probe 100 to which the electric cable 120 is connected. In such embodiments, the current-carrying wiring may be connected both the electrodes in the clamshell 116, such that current can flow into the first electrode 128 from an active wire (e.g., a wire carrying current connected to a generator), through anatomical tissue proximate the probe 100, into the second electrode 132, and from the second electrode 132 back out of the probe through a return wire (e.g., a wire that carries current back to the generator).

The electric cable 120 may also carry or pass a thermocouple 144 into the probe 100. The thermocouple 144 may be or comprise one or more sensors or thermally-sensitive material that measure the temperature of the electrodes of the probe 100. In some embodiments, each electrode of the probe 100 may be monitored by a separate thermocouple. The thermocouple 144 may, based on the heating experienced by the electrodes 128, 132 during operation, generate one or more measurements the represent the temperature of the electrodes 128, 132. In some embodiments, the thermocouple 144 may extend from the electric cable 120 into the clamshell 116, and may be threaded through or otherwise be disposed in the interior of the probe 100. As illustrated in FIGS. 1C and 1E, the thermocouple 144 may run along the length of the probe 100 from the proximal end 104 into the distal end 108 to measure the temperature of the electrodes. In some embodiments, the thermocouple 144 may be or comprise a type K thermocouple (e.g., a thermocouple with a temperature range of about 0 degrees Celsius (° C.) to about 1260° C.), a type J thermocouple (e.g., a thermocouple with a temperature range of about 0° C. to about 760° C.), a type N thermocouple (e.g., a thermocouple with a temperature range of about 0° C. to about 1260° C.), a type B thermocouple (e.g., a thermocouple with a temperature range of about 870° C. to about 1700° C.), a type E thermocouple (e.g., a thermocouple with a temperature range of about 0° C. to about 870° C.), a type R thermocouple (e.g., a thermocouple with a temperature range of about 500° C. to about 1500° C.), a type S thermocouple (e.g., a thermocouple with a temperature range of about 500° C. to about 1500° C.), or a type C thermocouple (e.g., a thermocouple with a temperature range of about 0° C. to about 2300° C.). The different types of thermocouples listed above may have different compositions of materials, such that the thermocouple can function at the specified temperature range while capturing temperature measurements. In some embodiments, the type of thermocouple may be selected based on the type of surgery, the anticipated temperature of the electrodes, the anticipated amount of time needed to ablate anatomical tissue, the surgical plan, combinations thereof, and the like. In some embodiments, the probe 100 may comprise multiple thermocouples to measure the temperature of one or more components (e.g., the electrodes of the probe 100).

The coolant input conduit 126 and the coolant output conduit 124 may comprise a coolant input channel 152 and a coolant output channel 156, respectively. The coolant input channel 152 may be or comprise a hollow, waterproof tube that carries fresh coolant (e.g., water, saline, etc.) into the probe 100 to cool (e.g., absorb heat from) the first electrode 128 and the second electrode 132. The coolant output channel 156 may be or comprise a hollow, waterproof tube that transfers spent coolant (e.g., coolant that has absorbed heat from the first electrode 128 and/or the second electrode 132) out of the probe 100. In some embodiments, the coolant input channel 152 and/or the coolant output channel 156 may extend through the elongated shaft 112 and into the distal end 108 of the probe 100. In other embodiments, the coolant input channel 152 may extend into the distal end 108 of the probe 100, while the coolant output channel 156 extends partially into the elongated shaft 112. In such embodiments, the coolant output channel 156 may exert a negative pressure and/or suction force to extract the coolant dispensed by the coolant input channel 152 in the distal end 108 of the probe 100 after the coolant has cooled the first electrode 128 and/or the second electrode 132.

FIG. 1B illustrates a detailed perspective view of the distal end 108 of the probe 100. The distal end 108 may include the first electrode 128, the second electrode 132, an insulation layer 136, a protective barrier 140, and the thermocouple 144.

The first electrode 128 and the second electrode 132 may conduct electrical current (e.g., an RF current) through proximal anatomical tissue thereto in order to ablate the anatomical tissue. The first electrode 128 may be, for example, an active electrode configured to receive current from current-carrying wire and carry the current to the surgical site at the distal end 108 of the probe 100. The second electrode 132 may be, for example, a return electrode configured to receive the current after the current has passed through the anatomical tissue at the surgical site and carry the current back through wiring to the generator or other power source to complete the circuit. In some embodiments, the first electrode 128 and/or the second electrode 132 may be or comprise hollow cylindrical tubes (e.g., stainless steel tubing, spring steel tubing, tubing comprising other metal alloys, etc.) extending from the proximal end 104 of the probe 100 (e.g., from within the interior cavity 118 of the clamshell 116) toward the distal end 108 of the probe 100. The first electrode 128 and/or the second electrode 132 may extend through a hollow interior of the elongated shaft 112. In some embodiments, the first electrode 128 may extend further toward the distal end 108 of the probe 100 than the second electrode 132.

In some embodiments, an insulation layer 136 may be disposed at least partially around the first electrode 128 or, more generally, between the first electrode 128 and the second electrode 132. The insulation layer 136 may be or comprise insulative material (e.g., plastic, PVC, Teflon, rubber, combinations thereof, etc.) capable of preventing current from passing from the first electrode 128 to other components of the probe 100. Additionally or alternatively, the insulation layer 136 may electrically separate the first electrode 128 and the second electrode 132, such that current passes through proximal anatomical tissue when travelling from the first electrode 128 to the second electrode 132 (or vice versa).

The protective barrier 140 may be or comprise insulative material, such as Polyimide (PI) heat shrinkable material that protects the second electrode 132 (e.g., prevents patient anatomy or other anatomical tissues from contacting the second electrode 132). The protective barrier 140 may be disposed at least partially around the second electrode 132 or, more generally, between the second electrode 132 and the elongated shaft 112. The protective barrier 140 may cover the portion of the second electrode 132 disposed outside the clamshell 116, which may beneficially increase safety and provide an insulative layer that can prevent or mitigate electric shock in the event the physician contact the second electrode 132 when the generator or power source is on.

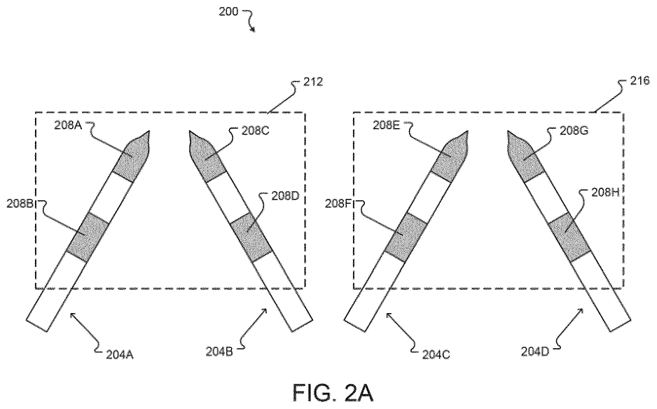

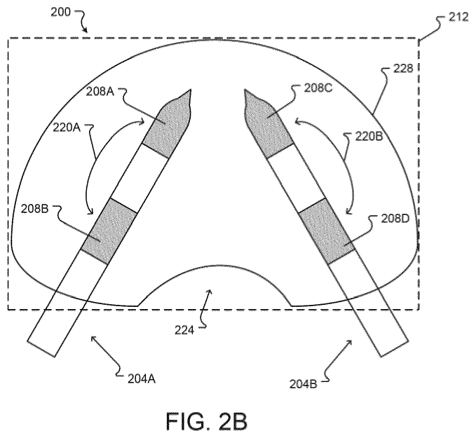

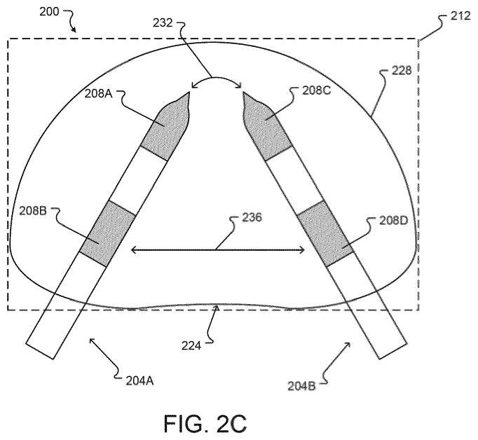

FIGS. 2A-2C illustrate aspects of an ablation system 200 in accordance with at least one embodiment of the present disclosure. The ablation system 200 may be used to ablate anatomical tissue. The ablation system 200 includes a first probe 204A, a second probe 204B, a third probe 204C, and a fourth probe 204D (collectively, a plurality of probes 204A-204D). In some embodiments, each probe of the plurality of probes 204A-204D may be similar to or the same as the probe 100. Each probe of the plurality of probes 204A-204D includes two electrodes. The first probe 204A includes a first electrode 208A and a second electrode 208B; the second probe 204B includes a third electrode 208C and a fourth electrode 208D; the third probe 204C includes a fifth electrode 208E and a sixth electrode 208F; and the fourth probe 204D includes a seventh electrode 208G and an eighth electrode 208H. The first probe 204A and the second probe 204B may be used to ablate a first ablation site 212, while the third probe 204C and the fourth probe 204D may be used to ablate a second ablation site 216. In some embodiments, the ablation system 200 may comprise an additional or alternative number of probes (e.g., 6 probes, 8 probes, etc.).

As shown in FIG. 2B, the first probe 204A and the second probe 204B may be used to ablate anatomical tissue in the first ablation site 212 using coaxial ablation. In coaxial ablation, current may flow from the first electrode 208A to the second electrode 208B (or vice versa) on the first probe 204A as depicted by arrows 220A, and current may also flow from the third electrode 208C to the fourth electrode 208D (or vice versa) on the second probe 204B, as indicated with arrows 220B. The current may pass through the anatomical tissue proximate the first probe 204A and the second probe 204B, ablating the anatomical tissue. The coaxial ablation of the first probe 204A and the second probe 204B may result in an ablation zone 228. The ablation zone 228 may represent the area of the first ablation site 212 where the anatomical tissue has been ablated. In some embodiments, the coaxial ablation may result in a gap 224 in the ablation zone 228. The gap 224 may include areas of the first ablation site 212 where anatomical tissue remains unablated. In other words, the coaxial ablation performed by the first probe 204A and the second probe 204B may result in areas in the first ablation site 212 where anatomical tissues that should be ablated are not ablated, such as when the anatomical tissues received insufficient current and/or heat to result in ablation. The gap 224 may result, for example, due to the current flow in the coaxial ablation mode of the first probe 204A and/or the second probe 204B being unable to reach the anatomical tissue in the gap 224.

As shown in FIG. 2C, the first probe 204A and the second probe 204B may be used to ablate anatomical tissue in the first ablation site 212 using cross-probe ablation. In cross-probe ablation, current may flow from the first electrode 208A to the third electrode 208C (or vice versa) as illustrated by arrows 232, and/or from the second electrode 208B to the fourth electrode 208D (or vice versa) as illustrated by arrows 236. In some embodiments, the current may be alternated between flowing from the first electrode 208A and the third electrode 208C (or vice versa) to flowing between the second electrode 208B and the fourth electrode 208D (or vice versa). Stated differently, the current flowing across the probes 204A, 204B may alternate such that only one electrode on each probe is used at any given time. In some embodiments, the current may flow from the first electrode 208A to the fourth electrode 208D (or vice versa), and/or from the second electrode 208B to the third electrode 208C (or vice versa). The cross-probe ablation of the first probe 204A and the second probe 204B may result in an ablation zone 228. The ablation zone 228 may represent the area of the first ablation site 212 where the anatomical tissue proximate the first probe 204A and the second probe 204B, and between the first probe 204A and the second probe 204B, has been ablated. As shown in FIG. 2C, the use of cross-probe ablation may reduce the gap 224 in the ablation zone 228. In other words, the use of cross-probe ablation may result in a larger ablation zone 228 than when using coaxial ablation.

In some embodiments, the first probe 204A and/or the second probe 204B may perform retract ablation. In retract ablation, the first probe 204A and/or the second probe 204B may ablate anatomical tissue adjacent to the first probe 204A and/or the second probe 204B (e.g., two millimeters, three millimeters, etc.) along the trajectory of the entry and exit path of the first probe 204A and/or the second probe 204B. In other words, the retract ablation may be performed after a coaxial and/or a cross-probe ablation, when the physician is retracting the first probe 204A and/or the second probe 204B from the surgical site. The retract ablation may facilitate the removal of the first probe 204A and/or the second probe 204B from the surgical site by ablating tissues that may have shifted to impede the exit path of the first probe 204A and/or the second probe 204B. In some embodiments, the retract ablation may be performed without cooling the probes 204A, 204B during the ablation. For example, the probes 204A, 204B may conduct current for a time short enough in duration, and/or ablate such a small amount of anatomical tissue, that the use of coolant would be unnecessary.

In some embodiments, the coaxial ablation, the cross-probe ablation, and/or the retract ablation performed by the first probe 204A and/or the second probe 204B may be controlled using power control, temperature control, and/or impedance control. The use of power control, temperature control, and/or impedance control may enable a physician to perform ablation of a surgical site while controlling the amount of current applied by the first probe 204A and/or the second probe 204B. Such control may reduce the probability of unintended damage to anatomical tissues or structures proximate the surgical site.

Power control may include applying a constant energy (e.g., 4 Watts (W)) from the first probe 204A and/or the second probe 204B to a surgical site for a predetermined amount of time. While the energy is applied, the temperature of the electrodes, the surgical site, and/or the like may be monitored with, for example, one or more thermocouples that generate a temperature measurement. When the measured temperature meets or exceeds a threshold value (which may be a predetermined value), the amount of energy (e.g., the magnitude of current) applied may be adjusted. For example, when the temperature exceeds the threshold value, and the energy applied by the probe may be decreased or discontinued. In some embodiments, the meeting or exceeding of the threshold value may increase the likelihood of charring of the surgical site. In some embodiments, the ablation system 200 may generate an alert to warn the physician that the temperature has met or exceeded the threshold value, which may indicate that the surgical site is at an increased likelihood of charring. Power control may be used for the first probe 204A and/or the second probe 204B during the performance of coaxial ablation, cross-probe ablation, and/or retract ablation. In one embodiment, the operation of the second electrode 208B and the fourth electrode 208D during cross-probe ablation may be monitored using power control.

Temperature control may use temperature measurements as a predictor of the amount of energy needed to achieve ablation. Temperature control may include modulating power applied from the first probe 204A and/or the second probe 204B to meet a target temperature of the surgical site. For example, a target temperature of the surgical site may be 70 degrees Celsius (° C.), and the first probe 204A and/or the second probe 204B may be inserted at 37° C. The power supplied by the probes 204A, 204B may be modulated (e.g., increased or decreased in a stepwise manner) based on the monitored temperature until the probes reach 70° C. In other words, temperature may drive the amount of energy delivered by the probes 204A, 204B. In some embodiments, the temperature may be monitored by one or more thermocouples. Temperature control may be used for the first probe 204A and/or the second probe 204B during the performance of coaxial ablation, cross-probe ablation, and/or retract ablation. For example, retract ablation may utilize temperature control. Additionally or alternatively, the operation of the first electrode 208A and the third electrode 208C during cross-probe ablation may be monitored using temperature control.

Impedance control may use impedance measurements to modulate the power supplied by the probes to meet a predetermined impedance value. The impedance may be measured, for example, by passing a small current between the electrodes of a probe (e.g., the first electrode 208A and the second electrode 208B of the first probe 204A), measuring voltages at the electrodes, and using the voltages and current to determine an impedance. In some embodiments, the impedance may be measured over a set of samples (e.g., 200 samples, 400 samples, 500 samples, etc.), and the determined impedance for each sample may be averaged to determine an average impedance. The determined impedance may be compared to the predetermined impedance value, and the power may be adjusted accordingly. For example, when the determined impedance is above the predetermined impedance threshold, the power may be increased to increase the temperature of the surgical site. Impedance control may be used for the first probe 204A and/or the second probe 204B during the performance of coaxial ablation, cross-probe ablation, and/or retract ablation.

While the first probe 204A and the second probe 204B are discussed above in FIGS. 2B and 2C, it is to be understood that the discussion also applies similarly or the same to the third probe 204C and the fourth probe 204D in ablating the second ablation site 216, as well as to any other probe or group of probes in the ablation system 200.

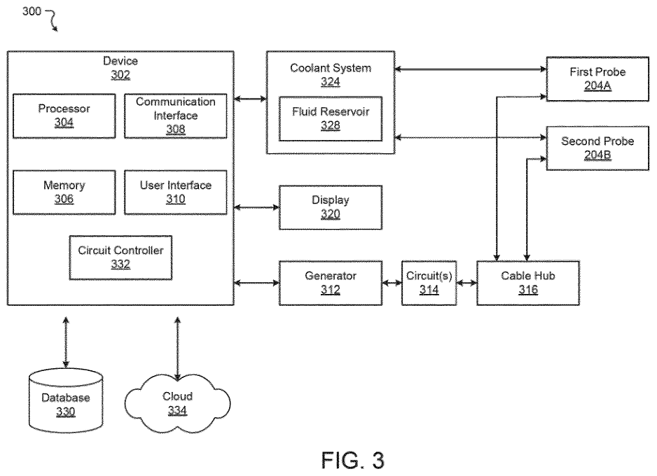

FIG. 3 depicts a block diagram of a system 300 according to at least one embodiment of the present disclosure. The system 300 may be used to control ablation of anatomical tissue using probes, enable user interaction and control of the probes, and/or to carry out one or more aspects of one or more of the methods disclosed herein. The system 300 comprises the first probe 204A, the second probe 204B, a device 302, a generator 312, one or more circuits 314, a cable hub 316, a display 320, a coolant system 324, a database 330, and a cloud or other network 334. In some embodiments, the system 300 may comprise additional or alternative components to those depicted in FIG. 3. For example, the system 300 may include the third probe 204C and the fourth probe 204D.

The device 302 comprises a processor 304, a memory 306, a communication interface 308, a user interface 310, and a circuit controller 332. In some embodiments, the device 302 may comprise more or fewer components than those depicted in FIG. 3.

The processor 304 of the device 302 may be any processor described herein or any similar processor. The processor 304 may be configured to execute instructions stored in the memory 306, which instructions may cause the processor 304 to carry out one or more computing steps utilizing or based on data received from the first probe 204A, the second probe 204B, the generator 312, the circuits 314, the cable hub 316, the coolant system 324, the database 330, and/or the cloud 334.

The memory 306 may be or comprise RAM, DRAM, SDRAM, other solid-state memory, any memory described herein, or any other tangible, non-transitory memory for storing computer-readable data and/or instructions. The memory 306 may store information or data useful for completing, for example, any step of the methods described herein, or of any other methods. The memory 306 may store, for example, instructions that support one or more functions of the first probe 204A and/or the second probe 204B. For instance, the memory 306 may store content (e.g., instructions) that, when executed by the processor 304, enable cooling of the first probe 204A and the second probe 204B and/or ablation (e.g., coaxial ablation, cross-probe ablation, retract ablation, etc.) with the first probe 204A and/or the second probe 204B. Such content, if provided as in instruction, may, in some embodiments, be organized into one or more applications, modules, packages, layers, or engines. Alternatively or additionally, the memory 306 may store other types of content or data that can be processed by the processor 304 to carry out the various method and features described herein. Thus, although various contents of memory 306 may be described as instructions, it should be appreciated that functionality described herein can be achieved through use of instructions, algorithms, data, and/or the like. The data, algorithms, and/or instructions may cause the processor 304 to manipulate data stored in the memory 306 and/or received from or via the first probe 204A, the second probe 204B, the generator 312, the circuits 314, the cable hub 316, the coolant system 324, the database 330, and/or the cloud 334.

The communication interface 308 may be used for receiving data or other information from an external source (such as the generator 312, the display 320, the coolant system 324, the database 330, the cloud 334, and/or any other system or component not part of the system 300), and/or for transmitting instructions or other information to an external system or device (e.g., another device 302, the generator 312, the display 320, the coolant system 324, the database 330, the cloud 334, and/or any other system or component not part of the system 300). The communication interface 308 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 308 may be useful for enabling the device 302 to communicate with one or more other processors 304 or computing devices 302, whether to reduce the time needed to accomplish a computing-intensive task or for any other reason.

The user interface 310 may be or comprise a keyboard, mouse, trackball, monitor, television, screen, touchscreen, and/or any other device for receiving information from a user and/or for providing information to a user. The user interface 310 may be used, for example, to receive a user selection or other user input regarding any step of any method described herein. Notwithstanding the foregoing, any required input for any step of any method described herein may be generated automatically by the system 300 (e.g., by the processor 304 or another component of the system 300) or received by the system 300 from a source external to the system 300. In some embodiments, the user interface 310 may be useful to allow a surgeon or other user to modify instructions to be executed by the processor 304 according to one or more embodiments of the present disclosure, and/or to modify or adjust a setting of other information displayed on the user interface 310 or corresponding thereto.

Although the user interface 310 is shown as part of the device 302, in some embodiments, the device 302 may utilize a user interface 310 that is housed separately from one or more remaining components of the device 302. For example, the user interface 310 (or more generally the device 302) may be disposed within the display 320. In some embodiments, the user interface 310 may be located proximate one or more other components of the device 302, while in other embodiments, the user interface 310 may be located remotely from one or more other components of the device 302.

The generator 312 may generate current (e.g., RF current, an RF alternating current, etc.) that is passed into the first probe 204A and/or the second probe 204B through use of the circuits 314. The circuits 314 may include one or more electrical components (e.g., batteries, resistors, capacitors, inductors, etc.) that facilitate the generation or modulation of current carried to the first probe 204A and/or the second probe 204B. For example, the generator 312 may generate a fixed current, and the circuits 314 may be controlled by the circuit controller 332 (e.g., the circuit controller 332 may cause switches in the circuits 314 to open or close to adjust current amplification) to generate a desired current that is passed through the first probe 204A and/or the second probe 204B. Additionally or alternatively, the circuits 314 may be cyclable such that the first probe 204A and/or the second probe 204B can switch between different modes of operation. For example, the user may desire to switch from coaxial ablation to cross-probe ablation, and may enter a command into the system 300 (e.g., via the user interface 310) to switch to cross-probe ablation. The circuit controller 332 may then cause the current flowing from the generator 312 to flow differently within the circuits 314, such that cross-probe ablation is performed instead of coaxial ablation. In this example, the current originally flowing out of the first electrode 208A and into the second electrode 208B may switch to flowing into the third electrode 208C. Similarly, current may flow out of the second electrode 208B and into the fourth electrode 208D.

In some embodiments, the circuit controller 332 may control the circuits 314 based on inputs from the user, instructions stored in the database 330, or the like. For example, the user may desire a first current, and may provide an input through the user interface 310 to the circuit controller 332 that instructs the circuit controller 332 to generate the first current. The circuit controller 332 may (e.g., by executing instructions stored in the memory 306) cause the generator 312 to generate the first current. Additionally or alternatively, the circuit controller 332 may cause the generator 312 to generate a current, and may adjust the circuits 314 (e.g., by passing the current through one or more current amplifiers) such that the current passed into the first probe 204A and/or the second probe 204B is the first current.

In some embodiments, one or more components of the system 300 (e.g., the device 302 and/or components thereof, the display 320, the database 330, etc.) may be housed within the generator 312. In such embodiments, a physician or other user may be able to control the first probe 204A, the second probe 204B, the circuits 314, the coolant system 324, combinations thereof, and/or the like from the generator 312. For example, the generator 312 may include the device 302 and the display 320, such that the physician, through use of the user interface 310, can control the type of ablation performed by the first probe 204A and/or the second probe 204B, when the coolant system 324 is operating, the type of control (e.g., power control, temperature control, impedance control, etc.) used in operating the first probe 204A and/or the second probe 204B, combinations thereof, and the like.

The cable hub 316 may connect the circuits 314 to the first probe 204A and/or to the second probe 204B. The cable hub 316 may comprise a plurality of ports that each connect to a different probe. For example, the cable hub 316 may include four ports, with each port connected respectively to the first probe 204A, the second probe 204B, the third probe 204C, and the fourth probe 204D. The cable hub 316 may be configured to pass current received from the generator 312 into the plurality of probes 204A-204D. In some embodiments, the circuits 314 may be disposed within the cable hub 316, such that current generated by the generator 312 is split at the cable hub 316 by the circuits 314, and passed into one or more of the plurality of probes 204A-204D. The cable hub 316 may also include a plurality of thermocouple ports, with each thermocouple port couplable to a thermocouple. Each thermocouple attached to the cable hub 316 may be similar to the thermocouple 144, and may be disposed proximate a probe or an electrode of the probe to generate a temperature measurement. The cable hub 316 may be connected to or otherwise communicate with the device 302, and may pass along temperature measurements generated by the thermocouples.

The display 320 may be or comprise a screen or touchscreen that renders information related to the ablation for the user to view. In some embodiments, the user may be able to control one or more components of the system 300 through the display 320 (e.g., the display 320 may comprise the user interface 310). In one embodiment, the display 320 and the generator 312 may be disposed in the same housing. The type of information rendered to the display 320 is in no way limited, and some examples of information related to the surgery include an amount of power supplied to the first probe 204A and/or the second probe 204B (or, more specifically, to the first electrode 208A, the second electrode 208B, the third electrode 208C, and/or the fourth electrode 208D); information about a temperature of the first electrode 208A; information about a temperature of the second electrode 208B; information about a measured impedance between the first electrode 208A and the third electrode 208C and/or information about a measured impedance between the second electrode 208B and the fourth electrode 208D (such as when impedance control is used for the probes 204A, 204B); combinations thereof; and the like.

The coolant system 324 may control the cooling of the first probe 204A and/or the second probe 204B, and includes a fluid reservoir 328. The fluid reservoir 328 may have one or more containers that house one or more coolants (e.g., water, saline, etc.). One or more pumps may be controlled to pump the coolant into the first probe 204A and/or the second probe 204B. The coolant may be fluidically communicated to the first probe 204A and/or the second probe 204B through one or more fluid conduits (e.g., through the coolant input channel 152 and the coolant output channel 156). In some embodiments, the coolant system 324 may comprise a suction mechanism that can be turned on (e.g., begin generating a vacuum to remove coolant from the first probe 204A and/or the second probe 204B) when coolant is supplied to the first probe 204A and/or the second probe 204B. As such, coolant may be dispensed into a distal end of the first probe 204A and/or the second probe 204B, such as by the coolant input channel 152, and may be removed from the distal end by the coolant output channel 156. In one embodiment, the coolant system 324 may cause the coolant to be pumped into the first probe 204A to cool the probe. The coolant may then be pumped from the first probe 204A and into the second probe 204B to cool the second probe. The coolant may then be pumped back into a separate container in the fluid reservoir 328. In some embodiments, the coolant system 324 may be controlled by the device 302 and/or by input commands by the user (e.g., via the user interface 310).

The database 330 may store information related to one or more surgical plans (e.g., information related to the type of tissue to be ablated, the position and orientation of one or more anatomical elements of a patient, etc.); information related to the first probe 204A and/or the second probe 204B (e.g., a model type, a recommended operating temperature or power range, etc.); and/or any other useful information. The database 330 may be configured to provide any such information to the device 302 or to any other device of the system 300 (e.g., to the generator 312, to the circuits 314, to the display 320, to the coolant system 324) or external to the system 300, whether directly or via the cloud 334. In some embodiments, the database 330 may be or comprise part of a hospital image storage system, such as a picture archiving and communication system (PACS), a health information system (HIS), and/or another system for collecting, storing, managing, and/or transmitting electronic medical records including image data.

The cloud 334 may be or represent the Internet or any other wide area network. The device 302 may be connected to the cloud 334 via the communication interface 308, using a wired connection, a wireless connection, or both. In some embodiments, the device 302 may communicate with the database 330 and/or an external device via the cloud 334.

The system 300 or similar systems may be used, for example, to carry out one or more aspects of any of the methods described herein. The system 300 or similar systems may also be used for other purposes.

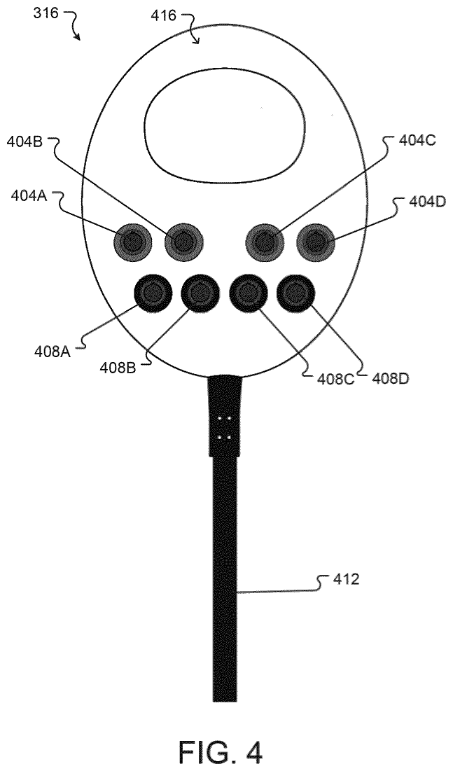

FIG. 4 depicts aspects of the cable hub 316 in accordance with embodiments of the present disclosure. The cable hub 316 comprises a plurality of probe ports 404A-404D, a plurality of thermocouple ports 408A-408D, an output port 412, and a handle area 416.

The probe ports 404A-404D may each provide a location for the plurality of probes 204A-204D to connect, to connect the plurality of probes 204A-204D to the system 300 and/or components thereof. In other words, the first probe 204A may be connectable to a first probe port 404A, the second probe 204B may be connectable to a second probe port 404B, the third probe 204C may be connectable to a third probe port 404C, and the fourth probe 204D may be connectable to a first probe port 404D. The connection of the plurality of probes 204A-204D to the cable hub 316 may enable the system 300 to monitor and/or control the plurality of probes 204A-204D during the course of a surgery or surgical procedure. For example, the output port 412 may connect the cable hub 316 to the circuits 314, the generator 312, or the like, such that the current supplied to the plurality of probes 204A-204D can be adjusted (e.g., based on instructions stored in the memory 306 and executed by the circuit controller 332). In some embodiments, the circuits 314 may be disposed within the cable hub 316, such that current (e.g., alternating RF current) generated by the generator 312 can be split by the circuits 314 and output through the appropriate port 404A-404D. For example, the user may want to perform coaxial ablation using the third probe 204C and the fourth probe 204D. In this case, the generator 312 may generate a current that is received at the output port 412. The circuits 314 may then direct the current out of the third probe port 404C and the fourth probe port 404D, such that only the third probe 204C and the fourth probe 204D receive current.

In some embodiments, when the user desires the plurality of probes 204A-204D to operate in different ablation modes, the circuits 314 may adjust how the current flows through the cable hub 316 and into the plurality of probes 204A-204D to match the ablation mode desired by the user. For example, the user may wish to switch the first probe 204A and the second probe 204B into a cross-probe ablation mode from a coaxial ablation mode (e.g., the first probe 204A and the second probe 204B were both independently operating in a coaxial ablation mode). In this example, the user may provide an input (e.g., via the user interface 310) to cause the first probe 204A and the second probe 204B to operate in the cross-probe mode, and the circuits 314 may cause the current flow to the first probe 204A and the second probe 204B to change to perform cross-probe ablation based on, for example, signals received at the cable hub 316 via the output port 412. For instance, current may be flowing from the first electrode 208A to the second electrode 208B and from the third electrode 208C to the fourth electrode 208D when the first probe 204A and the second electrode 208B operate in the coaxial ablation mode, and the circuits 314 may change the current flow to perform cross-probe ablation, such that current flows from the first electrode 208A to the third electrode 208C and/or from the second electrode 208B to the fourth electrode 208D.

The thermocouple ports 408A-408D may each be connectable to a thermocouple, which may be similar to or the same as the thermocouple 144. The thermocouples may be placed proximate the probes or electrodes of the probes to generate temperature measurements thereof. The measurements may be passed through the cable hub 316 and out of the output port 412. The output port 412 may be connected to, for example, the device 302, the generator 312, and/or any other component of the system 300. In some embodiments, the thermocouple readings generated by the thermocouples and passed through the thermocouple ports 408A-408D may be used to control the plurality of probes 204A-204D. For example, the temperature readings generated by the thermocouples may be used to perform temperature control of the first probe 204A and/or the second probe 204B to ensure that anatomical tissue proximate the first probe 204A and/or the second probe 204B does not overheat.

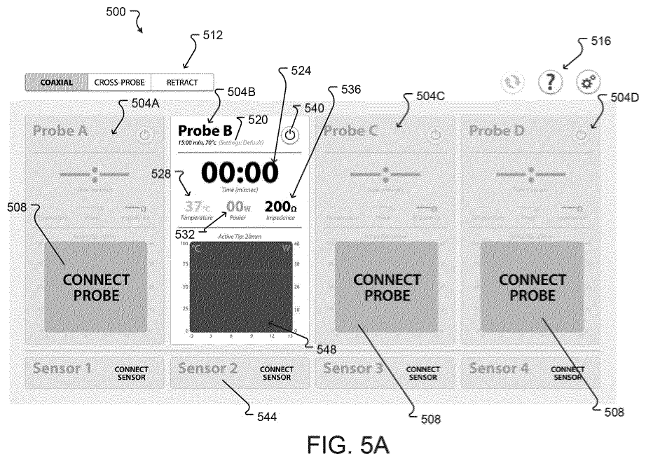

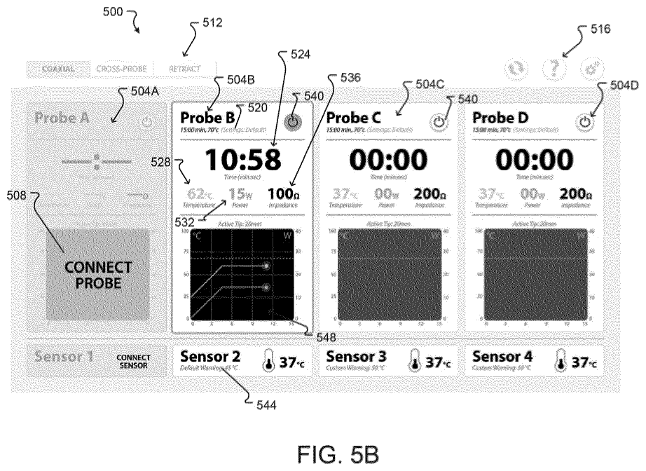

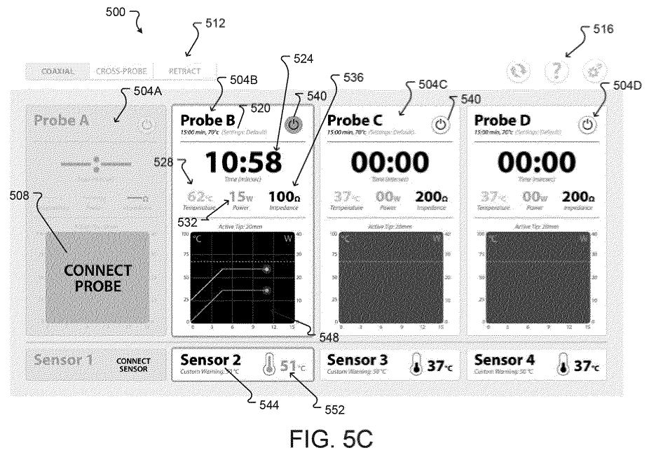

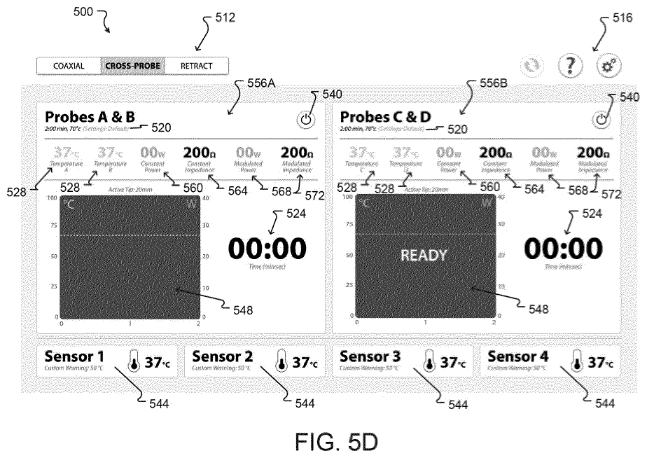

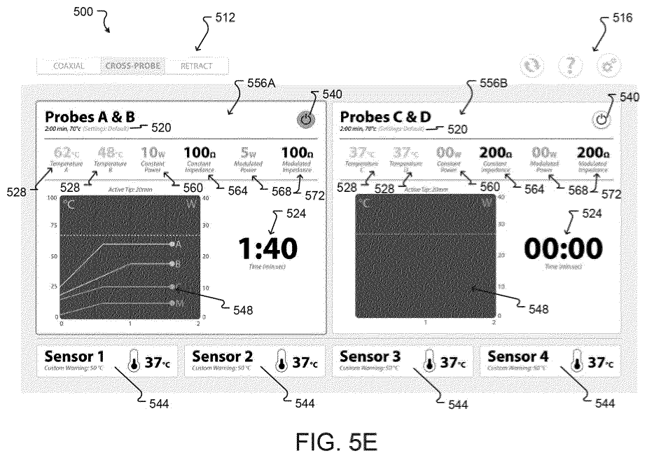

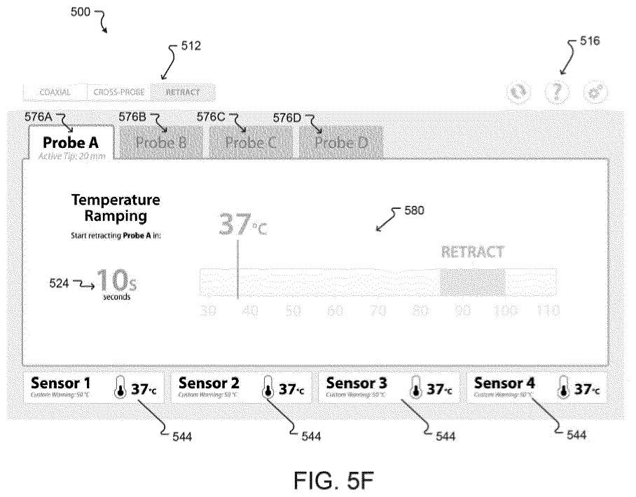

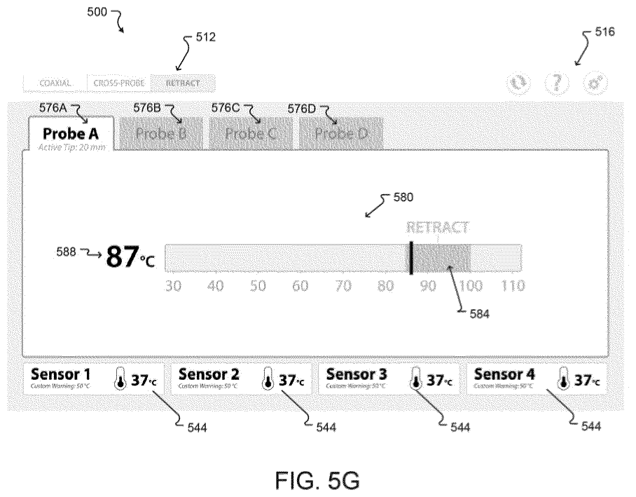

FIGS. 5A-5G illustrate aspects of a display 500 in accordance with embodiments of the present disclosure. The display 500 may, in some embodiments, be similar to or the same as the display 320. The display 500 comprises probe windows 504A-504D, probe modes 512, settings and help buttons 516, and sensor information 544.

The settings and help buttons 516 may provide buttons (e.g., buttons rendered on a touchscreen) that enable the user to adjust the settings of the display 500 (e.g., screen display settings such as brightness, position/orientation of the probe windows 504A-504D, etc.) and/or to access help in operating the display 500 (e.g., instructions rendered to the display 500 to assist the user in changing the mode of operation of the plurality of probes 204A-204D). The settings may allow the user to change the ablation settings for the plurality of probes 204A-204D. In some embodiments, the changes made by the user to the ablation settings may be reflected in the ablation settings 520 rendered to each probe window 504A-504D.

The probe windows 504A-504D may display information (e.g., a visual depiction) related to any one or more of the plurality of probes 204A-204D. For example, a first probe window 504A may display information related to the first probe 204A, a second probe window 504B may display information related to the second probe 204B, a third probe window 504C may display information related to the third probe 204C, and a fourth probe window may display information related to the fourth probe 204D. As illustrated in FIG. 5A, the second probe window 504B may display information about the second probe 204B, while the remaining windows may be greyed out. In this case, the second probe 204B may be connected to the generator 312 and capable of performing ablation, while the remaining probes may not be connected, or may be connected to the generator 312 but otherwise disabled or unable to perform ablation. As shown in FIG. 5B, the second probe window 504B, the third probe window 504C, and the fourth probe window 504D may all display probe information, while the first probe window 504A may be greyed out. In this example, the second probe 204B, the third probe 204C, and the fourth probe 204D may each be turned on or otherwise capable of performing ablation, while the first probe 204A may be turned off or otherwise not capable of performing ablation.

The probe windows 504A-504D may each include one or more renderings to display information related to the respective plurality of probes 204A-204D. As shown in FIG. 5A, the second probe window 504B may display one or more timers 524, temperature settings 528, power settings 532, impedance settings 536, a power button 540, and one or more graphs 548. Notwithstanding the foregoing, the probe windows 504A-504D may display additional or alternative information.

The probe mode 512 may depict the current operating mode of the probes. For example, FIGS. 5A-5C illustrate the display 500 when the plurality of probes 204A-204D are operating in a coaxial ablation mode, FIGS. 5D-5E illustrate the display 500 when the plurality of probes 204A-204D are operating in a cross-probe ablation mode, and FIGS. 5F-5G illustrate the display 500 when the plurality of probes 204A-204D are operating in a retract ablation mode. The user may be able to change the mode of operation of the plurality of probes 204A-204D by changing the probe mode 512 (e.g., by pressing the different modes on a touchscreen) or by providing an input (e.g., via the user interface 310) to the system 300 to change the mode in which one or more probes of the plurality of probes 204A-204D operate.

The timer 524 may indicate how much time remains for a given ablation. As shown in FIG. 5C, the second probe 204B may have 10 minutes and 58 seconds remaining for the coaxial ablation performed by the second probe 204B. When the timer reaches zero, the coaxial ablation procedure may end, and the system 300 or one or more components thereof (e.g., the circuit controller 332) may automatically disable current flow in the second probe 204B to stop the second probe 204B from ablating anatomical tissue.

The temperature settings 528, the power settings 532, and the impedance settings 536 may be based on target values that are predetermined and accessed from the database 330, based on the surgical plan, based on information the user inputs into the system 300 (e.g., via the user interface 310) to control the plurality of probes 204A-204D, combinations thereof, and the like. As shown in FIG. 5B, the temperature setting 528 may indicate that the temperature of the second probe 204B is 62° C. (e.g., based on a temperature measurement taken by a thermocouple disposed within or proximate the second probe 204B), the power setting 532 may indicate that the power currently applied to the second probe 204B is 15 W (e.g., based on the settings of the generator 312), and the impedance setting 536 may indicate that there is an impedance of 100 Ohms (Ω) between the third electrode 208C and the fourth electrode 208D (e.g., based on current supplied by the generator 312 and voltage measured across the electrodes). In some embodiments, the current values and the previously recorded values of the temperature settings 528, the power settings 532, and/or the impedance settings 536 may be displayed on the graph 548 that is rendered with the second probe window 504B.

The sensor information 544 may be based on temperature measurements taken by a thermocouple, such as the thermocouple 144. For example, the thermocouple may be disposed proximate the second probe 204B, and may monitor the temperature of the anatomical tissue being ablated. In some embodiments, the display 500 may render one or more warnings or alerts when a measurement of a parameter (e.g., temperature, power, impedance, etc.) meets or exceeds a predetermined threshold value. As shown in FIG. 5C, the sensor information 544 may indicate that the temperature measured by the temperature sensor is 51° C., and a warning 552 may be generated. The measurement of 51° C. may meet or exceed a predetermine threshold, and may indicate that the anatomical tissue proximate the thermocouple is receiving too much heat. In some embodiments, the warning 552 may be or comprise an audial warning (e.g., a beep, a siren, etc.), a visual indicator (e.g., the sensor information 544 may be rendered in a different color), combinations thereof, and the like.

The power button 540 may be a button that the user can press (e.g., on a touchscreen) to turn on the respective probe. In some embodiments, the power button 540 may provide a visual indicator that the probe is active. For example, and as shown in FIG. 5C, the second probe 204B may be powered on while the third probe 204C may be powered off, as shown by the different coloring of the power button 540 associated with the second probe window 504B and the power button 540 associated with the third probe window 504C.

FIGS. 5D-5E depicts aspects of the display 500 when the ablation system 200 is operated in a cross-probe ablation mode in accordance with embodiments of the present disclosure. The display 500 may comprise cross-probe windows 556A-556B, the probe mode 512, the settings and help buttons 516, and the sensor information 544.

A first cross-probe window 556A may be associated with the first probe 204A and the second probe 204B when the first probe 204A and the second probe 204B perform cross-probe ablation, while a second cross-probe window 556B may be associated with the third probe 204C and the fourth probe 204D when the third probe 204C and the fourth probe 204D perform cross-probe ablation. Similar to the probe windows 504A-504D for coaxial ablation, the cross-probe windows 556A-556B may comprise temperature settings 528, which may indicate the temperature of the first probe 204A and the second probe 204B. The cross-probe windows 556A-556B may also each comprise a constant power setting 560, a constant impedance setting 564, a modulated power setting 568, and a modulated impedance setting 572. The constant power setting 560 in the first cross-probe window 556A may indicate the amount of power supplied to the first probe 204A and/or the second probe 204B, while the constant impedance setting 564 in the first cross-probe window 556A may reflect the impedance between the first probe 204A and the second probe 204B (e.g., between the first electrode 208A and the third electrode 208C). The modulated power setting 568 in the first cross-probe window 556A may indicate the amount of modulated power applied to the first probe 204A (such as when the cross-probe ablation is monitored by temperature control). The modulated impedance setting 572 in the first cross-probe window 556A may indicate the amount of modulated impedance between the first probe 204A and the third probe 204C. Similar to in coaxial mode, the cross-probe windows 556A-556B may include graphs 548 that depict the previous temperature, power, and/or impedance values.

FIGS. 5F-5G depict aspects of the display 500 when the ablation system 200 is operated in a retract ablation mode in accordance with embodiments of the present disclosure. The ablation system 200 may operate the plurality of probes 204A-204D in retract mode when the ablation of anatomical tissue has been completed (e.g., in coaxial and/or cross-probe modes), and the plurality of probes 204A-204D are to be extracted from the surgical site. The display 500 includes retract probe windows 576A-576D which each respectively reflect information associated with the plurality of probes 204A-204D. The display 500 may comprise retract information 580, which may include a timer 524 and a temperature range 584. When in retract mode, any one or more probes of the plurality of probes 204A-204D may be ramped up to a predetermined temperature (e.g., using temperature control). When at the predetermined temperature, as illustrated in FIG. 5G, a temperature range 584 for the retract mode may be rendered to the first retraction probe window 576A, which may be associated with the first probe 204A. The first retraction probe window 576A may indicate a temperature 588 of the first probe 204A (e.g., 87° C.), and the first probe 204A may perform retract ablation to ablate anatomical tissue proximal the exit trajectory of the first probe 204A. After the ablation has been performed, the first probe 204A may be extracted. Similarly, any one or more of the second probe 204B, the third probe 204C, and/or the fourth probe 204D may perform retract ablation similar to or the same as the first probe 204A.

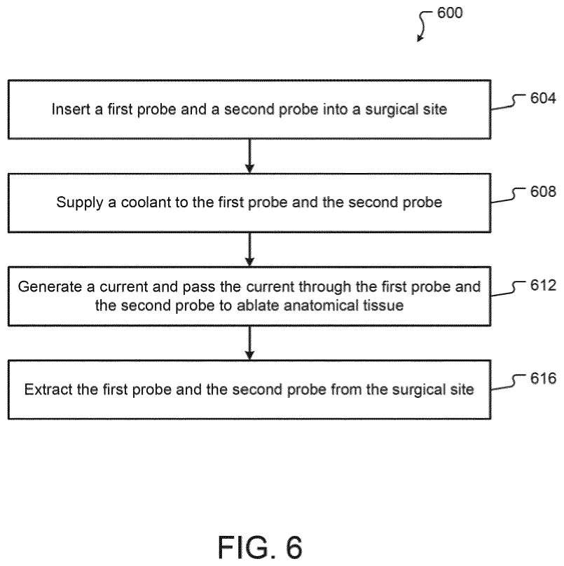

FIG. 6 depicts a method 600 that may be used, for example, to ablate anatomical tissue.

The method 600 (and/or one or more steps thereof) may be carried out or otherwise performed, for example, by at least one processor. The at least one processor may be the same as or similar to the processor(s) 304 of the device 302 described above. A processor other than any processor described herein may also be used to execute one or more steps of the method 600. The at least one processor may perform one or more steps of the method 600 by executing elements stored in a memory such as the memory 306. The elements stored in the memory and executed by the processor may cause the processor to execute one or more steps of a function as shown in method 600. One or more portions of a method 600 may be performed by the processor executing any of the contents of the memory.

The method 600 comprises inserting a first probe and a second probe into a surgical site (step 604). In some embodiments, the first probe may be similar to or the same as the first probe 204A, and the second probe may be similar to or the same as the second probe 204B. The surgical site may be, for example, a vertebra with anatomical tissue that is to be ablated by the first probe and the second probe. In some embodiments, the first probe and the second probe may be surgical inserted (e.g., by drilling pedicle tracks and inserting the probes into the vertebra through the tracks). The first probe may include first and second electrodes, while the second probe may include third and fourth electrodes. The first probe and the second probe may be configured to perform coaxial ablation, cross-probe ablation, and/or retract ablation.

The method 600 also comprises supplying a coolant to the first probe and the second probe (step 608). In some embodiments, the coolant may be provided by a coolant apparatus (e.g., the coolant system 324). The coolant apparatus may pump coolant into the first probe and/or the second probe while the first probe and/or second probe perform ablation to cool one or more electrodes of each probe. In some embodiments, the coolant may flow from a fluid reservoir (e.g., fluid reservoir 328) and into the first probe and/or the second probe via fluid conduits. In one embodiment, the coolant may flow from the fluid reservoir through both the first probe and the second probe. For example, coolant may flow into the first probe to cool the first and second electrodes of the first probe, and then into the second probe to cool the third and fourth electrodes of the second probe. The spent coolant may then flow back out of the second probe and into a container within the fluid reservoir.

The method 600 also comprises generating a current and passing the current through the first probe and the second probe to ablate anatomical tissue (step 612). The current may be generated using, for example, a generator such as the generator 312 or any other generator. The current generated may be sufficient to perform coaxial ablation, cross-probe ablation, and/or retract ablation by the first probe and the second probe. In some embodiments, the amount of current applied may be monitored using temperature control, power control, and/or impedance control.

The method 600 also comprises extracting the first probe and the second probe from the surgical site (step 616). The first probe and the second probe may be extracted from the surgical site once ablation has been performed to the surgical site. In some embodiments, retract ablation may be performed by the first probe and/or the second probe to clear away anatomical tissues disposed in the exit trajectory of the first probe and/or the second probe.

The present disclosure encompasses embodiments of the method 600 that comprise more or fewer steps than those described above, and/or one or more steps that are different than the steps described above.

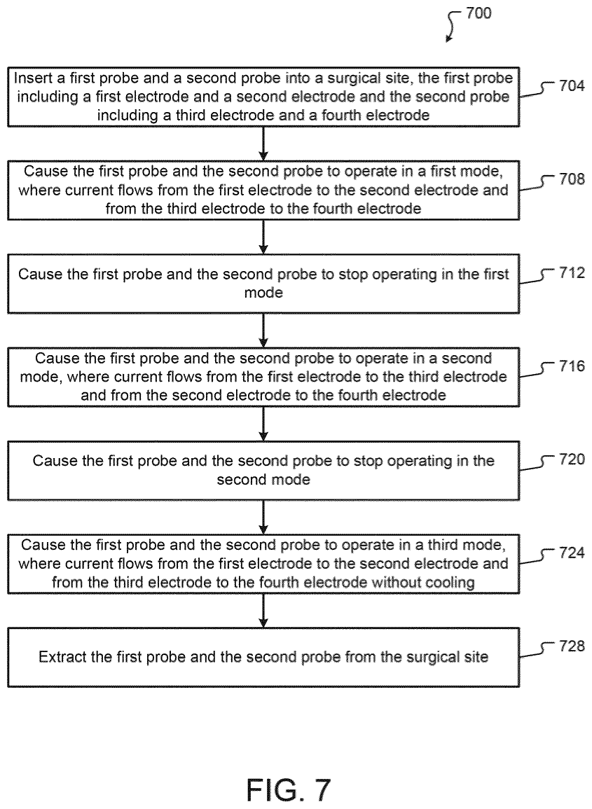

FIG. 7 depicts a method 700 that may be used, for example, to perform ablation of a surgical site with a set of probes capable of performing coaxial ablation, cross-probe ablation, and/or retract ablation.

The method 700 (and/or one or more steps thereof) may be carried out or otherwise performed, for example, by at least one processor. The at least one processor may be the same as or similar to the processor(s) 304 of the computing device 302 described above. A processor other than any processor described herein may also be used to execute one or more steps of the method 700. The at least one processor may perform one or more steps of the method 700 by executing elements stored in a memory such as the memory 306. The elements stored in memory and executed by the processor may cause the processor to execute one or more steps of a function as shown in method 700. One or more portions of a method 700 may be performed by the processor executing any of the contents of memory.

The method 700 comprises inserting a first probe and a second probe into a surgical site, the first probe including a first electrode and a second electrode and the second probe including a third electrode and a fourth electrode (step 704). In some embodiments, the step 704 may be similar to or the same as the step 604 of the method 600. The first probe (e.g., the first probe 204A) may include the first electrode and the second electrode (e.g., the first electrode 208A and the second electrode 208B), while the second probe (e.g., the second probe 204B) may include the third electrode and the fourth electrode (e.g., the third electrode 208C and the fourth electrode 208D). The first probe and the second probe may be configured to perform ablation in a plurality of different modes, such as coaxial ablation mode, cross-probe mode, and/or retract mode. In some embodiments, an additional or alternative number of probes may be inserted into the surgical site (e.g., four probes, six probes, eight probes, etc.).

The method 700 also comprises causing the first probe and the second probe to operate in a first mode, where current flows from the first electrode to the second electrode and from the third electrode to the fourth electrode (step 708). The first mode may be or comprise a coaxial ablation mode. In some embodiments, the first probe and the second probe may be controlled using a display, such as the display 320. While in coaxial ablation mode, one or more probe windows may be rendered to the display to display information related to the first probe and/or the second probe. In some embodiments, the probe windows may be similar to or the same as the probe windows 504A-504D. While in the coaxial ablation mode, one or more circuits (e.g., circuits 314) may control how current flows into the first probe and/or the second probe. In some embodiments, the coaxial ablation may be monitored using temperature, power, and/or impedance control.

The method 700 also comprises causing the first probe and the second probe to stop operating in the first mode (step 712). In some embodiments, it may be determined that coaxial ablation is to be discontinued. For example, the user may choose to turn off the coaxial ablation, or a user or system (e.g., the system 300 using the processor 304) may determine that the first probe and the second probe should operate in a different mode, such as a cross-probe ablation mode. The circuits may discontinue current flow to the first probe and the second probe, such that the first probe and the second probe no longer perform coaxial ablation.

The method 700 also comprises causing the first probe and the second probe to operate in a second mode, where current flows from the first electrode to the third electrode and from the second electrode to the fourth electrode (step 716). The second mode may be or comprise cross-probe ablation. While in the second mode, the circuits may adjust the current supplied by a generator (e.g., the generator 312), such that current flows from the first electrode of the first probe to the third electrode of the second probe (or vice versa), and/or from the second electrode of the first probe to the fourth electrode of the second probe (or vice versa). In some embodiments, the user may be able to monitor the cross-probe ablation using cross-probe ablation windows (e.g., cross-probe windows 556A-556B) rendered to the display. In some embodiments, the cross-probe ablation may be monitored using temperature, power, and/or impedance control.

The method 700 also comprises causing the first probe and the second probe to stop operating in the second mode (step 720). In some embodiments, the step 720 may be similar to or the same as the step 712. In some embodiments, the cross-probe ablation may end after a predetermined amount of time, or when the user (e.g., a physician) determines that there has been sufficient ablation of the surgical site. The circuits may discontinue current flow to the first probe and the second probe, such that the cross-probe ablation terminates.

The method 700 also comprises causing the first probe and the second probe to operate in a third mode, where current flows from the first electrode to the second electrode and from the third electrode to the fourth electrode without cooling (step 724). The third mode may be or comprise retract ablation. In some embodiments, the retract ablation may include the circuits causing current may flow similarly to coaxial ablation, but without the use of temperature, power, or impedance control. The retract ablation may terminate once the exit trajectories of the probes have been cleared of anatomical tissue, and/or after a predetermined amount of time. In some embodiments, the retract ablation may be monitored by the user or system based on information rendered to the display, such as retract probe windows (e.g., retract probe windows 576A-576D). After retract ablation ends, the circuits may discontinue and/or prevent current flow within the first probe and the second probe.

The method 700 also comprises extracting the first probe and the second probe from the surgical site (step 728). In some embodiments, the step 728 may be similar to or the same as the step 616 of the method 600. Once retract ablation has been performed by the first probe and the second probe, the first probe and the second probe may be extracted from the surgical site.

The present disclosure encompasses embodiments of the method 700 that comprise more or fewer steps than those described above, and/or one or more steps that are different than the steps described above.

As noted above, the present disclosure encompasses methods with fewer than all of the steps identified in FIGS. 6 and 7 (and the corresponding description of the methods 600 and 700), as well as methods that include additional steps beyond those identified in FIGS. 6 and 7 (and the corresponding description of the methods 600 and 700). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

Further disclosed herein is the subject-matter of the following clauses:
1. A device, comprising:
a first port and a second port each configured to respectively connect to a first probe and a second probe, wherein the first probe and the second probe are configured to perform bipolar ablation; and
a third port and a fourth port each configured to respectively connect to a third probe and a fourth probe, wherein the third probe and the fourth probe are configured to perform bipolar ablation.
2. The device of clause 1, further comprising:
an output port that connects two or more of the first port, the second port, the third port, and the fourth port to a radio frequency (RF) generator; and
a thermocouple port that is releasably connectable to a thermocouple.
3 The device of clause 2, wherein the RF generator comprises a display.
4. The device of clause 3, wherein the display is configured to render information received from at least one of the first probe, the second probe, the third probe, the fourth probe, and the thermocouple port.
5. The device of clause 4, wherein the information comprises at least one of a temperature measurement, a constant power measurement, a constant impedance measurement, a modulated power measurement, and a modulated impedance measurement.
6. The device of clause 3 or of any of clauses 3-5, wherein the display is configured to render information associated with at least one of a cross-ablation mode, a coaxial mode, and a retract mode.
7. The device of clause 6, wherein the first probe, when in the coaxial mode, performs bipolar ablation with a first and a second electrode each coaxially disposed on the first probe.
8. The device of clause 1 or of any of clauses 1-7, wherein two or more of the first probe, the second probe, the third probe, and the fourth probe are fluidically connected to a coolant pump.
9. A system, comprising:
a radio frequency (RF) generator; and
a hub port device comprising:
   a first port and a second port each configured to respectively connect to a first probe and a second probe, wherein the first probe and the second probe are configured to perform bipolar ablation; and
   a third port and a fourth port each configured to respectively connect to a third probe and a fourth probe, wherein the third probe and the fourth probe are configured to perform bipolar ablation.
10. The system of clause 9, wherein the hub port device further comprises:
a thermocouple port to which a thermocouple is releasably connectable.
11. The system of clause 9 or of any of clauses 9-10, wherein an output port connects the first port, the second port, the third port, and the fourth port to the RF generator.
12. The system of clause 9 or of any of clauses 9-11, further comprising:
a display in communication with the RF generator and the hub port device.
13. The system of clause 12, wherein the display is configured to render information received from at least one of the first probe, the second probe, the third probe, and the fourth probe.
14. The system of clause 13, wherein the information comprises at least one of a temperature measurement, a constant power measurement, a constant impedance measurement, a modulated power measurement, and a modulated impedance measurement.
15. The system of clause 12 or of any of clauses 12-14, wherein the display is configured to render at least one of a cross-ablation mode, a coaxial mode, and a retract mode.
16. The system of clause 15, wherein the first probe, when in the coaxial mode, performs bipolar ablation with a first and a second electrode each coaxially disposed on the first probe.
17. The system of clause 9 or of any of clauses 9-16, further comprising:
a coolant pump fluidically connectable to two or more of the first probe, the second probe, the third probe, and the fourth probe.
18. A system, comprising:
a radio frequency (RF) generator;
a display;
a hub port device comprising:
   a first port and a second port each configured to respectively connect to a first probe and a second probe, wherein the first probe and the second probe are configured to perform bipolar ablation; and
   a third port and a fourth port each configured to respectively connect to a third probe and a fourth probe, wherein the third probe and the fourth probe are configured to perform bipolar ablation;
a processor; and
a memory storing data thereon that, when processed by the processor, cause the processor to:
   cause the RF generator to generate an RF current passed into the first probe and the second probe;
   receive measurement data associated with the first probe and the second probe; and
   render the measurement data to the display.
19. The system of clause 18, wherein the measurement data comprises at least one of a temperature measurement, a constant power measurement, a constant impedance measurement, a modulated power measurement, and a modulated impedance measurement.
20. The system of clause 19, wherein the display is configured to render information about a coaxial mode, and wherein the first probe, when in the coaxial mode, performs RF ablation between a first and a second electrode each coaxially disposed on the first probe.

1-15. (canceled)

16. A device, comprising:
a first port and a second port each configured to respectively connect to a first probe and a second probe, wherein the first probe and the second probe are configured to perform bipolar ablation; and
a third port and a fourth port each configured to respectively connect to a third probe and a fourth probe, wherein the third probe and the fourth probe are configured to perform bipolar ablation.

17. The device of claim 16, further comprising:
an output port that connects two or more of the first port, the second port, the third port, and the fourth port to a radio frequency (RF) generator; and
a thermocouple port that is releasably connectable to a thermocouple.

18. The device of claim 17, wherein the RF generator comprises a display.

19. The device of claim 18, wherein the display is configured to render information received from at least one of the first probe, the second probe, the third probe, the fourth probe, and the thermocouple port.

20. The device of claim 19, wherein the information comprises at least one of a temperature measurement, a constant power measurement, a constant impedance measurement, a modulated power measurement, and a modulated impedance measurement.

21. The device of claim 18, wherein the display is configured to render information associated with at least one of a cross-ablation mode, a coaxial mode, and a retract mode.

22. The device of claim 21, wherein the first probe, when in the coaxial mode, performs bipolar ablation with a first and a second electrode each coaxially disposed on the first probe.

23. The device of claim 16, wherein two or more of the first probe, the second probe, the third probe, and the fourth probe are fluidically connected to a coolant pump.

24. A system, comprising:
a radio frequency (RF) generator; and
a hub port device comprising:
a first port and a second port each configured to respectively connect to a first probe and a second probe, wherein the first probe and the second probe are configured to perform bipolar ablation; and
a third port and a fourth port each configured to respectively connect to a third probe and a fourth probe, wherein the third probe and the fourth probe are configured to perform bipolar ablation.

25. The system of claim 24, wherein the hub port device further comprises:
a thermocouple port to which a thermocouple is releasably connectable.

26. The system of claim 24, wherein an output port connects the first port, the second port, the third port, and the fourth port to the RF generator.

27. The system of claim 24, further comprising:
a display in communication with the RF generator and the hub port device.

28. The system of claim 27, wherein the display is configured to render information received from at least one of the first probe, the second probe, the third probe, and the fourth probe.

29. The system of claim 28, wherein the information comprises at least one of a temperature measurement, a constant power measurement, a constant impedance measurement, a modulated power measurement, and a modulated impedance measurement.

30. The system of claim 27, wherein the display is configured to render at least one of a cross-ablation mode, a coaxial mode, and a retract mode.

31. The system of claim 30, wherein the first probe, when in the coaxial mode, performs bipolar ablation with a first and a second electrode each coaxially disposed on the first probe.

32. The system of claim 24, further comprising:
a coolant pump fluidically connectable to two or more of the first probe, the second probe, the third probe, and the fourth probe.

33. A system, comprising:
a radio frequency (RF) generator;
a display;
a hub port device comprising:
a first port and a second port each configured to respectively connect to a first probe and a second probe, wherein the first probe and the second probe are configured to perform bipolar ablation; and
a third port and a fourth port each configured to respectively connect to a third probe and a fourth probe, wherein the third probe and the fourth probe are configured to perform bipolar ablation;
a processor; and
a memory storing data thereon that, when processed by the processor, cause the processor to:
cause the RF generator to generate an RF current passed into the first probe and the second probe;
receive measurement data associated with the first probe and the second probe; and
render the measurement data to the display.

34. The system of claim 33, wherein the measurement data comprises at least one of a temperature measurement, a constant power measurement, a constant impedance measurement, a modulated power measurement, and a modulated impedance measurement.

35. The system of claim 34, wherein the display is configured to render information about a coaxial mode, and wherein the first probe, when in the coaxial mode, performs RF ablation between a first and a second electrode each coaxially disposed on the first probe.
